(19) United States
(12) Patent Application Publication
(10) Pub. No.: US 2026/0204391 A1
(43) Pub. Date: Jul. 16, 2026

(54) Task, Reminder, and AI-Companion System for Multiple Device Platforms with Advanced AI-Driven Interactions

(71) Applicants: Costin Tuculescu, Tustin, CA (US); Gerald Norton, Hoboken, NJ (US)

(72) Inventors: Costin Tuculescu, Tustin, CA (US); Gerald Norton, Hoboken, NJ (US)

(21) Appl. No.: 19/023,811

(22) Filed: Jan. 16, 2025

(51) Int. Cl. G16H 20/70; G06Q 10/1093

(52) U.S. Cl. CPC G16H 20/70; G06Q 10/1097

(57) ABSTRACT

A Task, Reminder, and AI-Companion System for Television with Advanced AI-Driven Interactions is disclosed. The system includes a central database and cloud-based scheduler that dispatches tasks to a device connected to a senior's television. Tasks may include medication reminders, telehealth appointments, or personalized AI conversations. An AI companion, leveraging Large Language Models (LLMs), provides natural language interaction, accepts voice or camera input, and logs user responses for caregiver review. By integrating familiar television interfaces and advanced AI capabilities, the invention enhances senior care through comprehensive task management, emotional engagement, and data-driven oversight.

### CROSS-REFERENCE TO RELATED APPLICATIONS

This application claims the benefit of priority of any applicable U.S. Provisional Patent Applications, including but not limited to:
a. U.S. Provisional Application No. 63/621,662 filed on Jan. 17, 2024 titled “Task and Reminder System for TV with AI-based User Interface.”
b. U.S. Provisional Application No. 63/621,697 filed on Jan. 17, 2024 titled “TV-Based AI Companion with Specific Task Execution.”

The entire disclosures of the above-referenced provisional applications are hereby incorporated by reference.

### BACKGROUND OF THE INVENTION

The present invention generally pertains to senior care technology—in particular, systems and methods that utilize artificial intelligence (AI) to provide task management, reminders, health monitoring, and interactive companionship. Such a solution offers a familiar interface, such as a television or tablet, to assist older adults in maintaining daily routines, medication schedules, social engagement, and overall well-being.

### Overview of Existing Challenges

In senior care, it is crucial to ensure adherence to routines, medication schedules, and social activities, as well as to address emotional and mental well-being. Traditional methods (e.g., phone calls, text messages, in-person checkups) can be intrusive, ineffective, or complicated for seniors. Many older adults may have difficulty navigating smartphones or computer interfaces, hindering timely access to reminders, social interactions, or telehealth services.

### Limitations of Existing Systems

Existing senior care technologies often exhibit a range of shortcomings that inhibit their usefulness and accessibility for older adults and individuals with disabilities. Additionally, very little consideration is given to the caregiver. Existing systems lack the ease of scheduling, the variety and quality of tasks and the data collection and review. The identification and alerting of red-flag responses are lacking in other systems.

Lack user-friendly interfaces specifically tailored to seniors (requiring complex phone or smartphone use). In many cases, seniors are asked to navigate smartphone apps or computer systems designed for younger, more tech-savvy audiences. This can create frustration and confusion, leading to abandonment of important health or social tools. Simple tasks like tapping a touchscreen or swiping through menus may be cumbersome for those with limited dexterity or unfamiliarity with digital interfaces. Consequently, essential reminders or notifications may go unnoticed or improperly acknowledged, resulting in gaps in care.

Fail to provide robust, AI-driven social and emotional engagement beyond basic reminders. While some systems offer simple alert mechanisms (e.g., “Take your medication now”), they typically do not engage the senior in a meaningful way. The emotional and social dimensions of senior care—such as friendly conversation, companionship, or empathetic check-ins—are often overlooked. Without conversational AI or personalized interaction, seniors may feel isolated, and their caregivers lack deeper insight into daily emotional states or concerns. As a result, these systems provide only a fraction of the support seniors truly need.

Offer limited customization for diverse senior needs (e.g., medication tracking versus emotional support). Every senior has a unique set of requirements—some may only need medication reminders, while others benefit from more frequent social interactions or guidance for physical therapy routines. Existing technologies often enforce rigid workflows that fail to adapt to changes in health status or personal preferences over time. By not allowing sufficient tailoring, these solutions may be too broad (causing users to ignore irrelevant prompts) or too narrow (missing key aspects of care). Over time, seniors and caregivers alike can grow frustrated by the system's inability to evolve alongside changing needs.

Underutilize common devices familiar to seniors—like televisions or simple display devices. Many modern solutions assume users will interact primarily through smartphones or computers, overlooking the potential of household devices that seniors find more comfortable and intuitive. Televisions, for instance, are ubiquitous in senior living spaces, yet few care systems fully leverage TV-based reminders or visually engaging content on a large screen. In senior living facilities, systems are ‘broadcast’ based. The senior has to have the TV on, turned to the broadcast channel. All users see the same information. By failing to tap into such a familiar channel, and customizing the experience for each user, existing solutions inadvertently force seniors onto platforms they find less approachable, reducing overall engagement and compliance.

Do not take into account diminished capabilities of seniors and the disabled to manipulate physical interfaces such as telephone push buttons or smartphone touch-screens. As people age or face various disabilities, fine-motor skills, grip strength, and general hand-eye coordination can decline. Conventional care technologies frequently rely on precise inputs—like pressing small buttons or navigating small on-screen controls—to register responses. This physically demanding method excludes many seniors who struggle with arthritis, tremors, or other impairments. In turn, these individuals either avoid using the system altogether or receive inadequate support because they cannot reliably interact with it.

Do not account for difficulties in responding in prescribed fashions, such as saying ‘yes’ or ‘no’. Many automated systems require specific answers to prompts—often limited to short voice inputs or button-press confirmations. Seniors who respond in more natural or free-form language (for example, “I did that already” instead of “yes”) may have their input unrecognized by the system. This mismatch between real human communication patterns and rigidly expected answers creates confusion, prevents accurate data logging, and can undermine trust in the system's usefulness. As a result, important reminders or queries may go unresolved, leading to less effective care

By not addressing these limitations, conventional senior care platforms fail to adequately meet the fundamental needs of older adults, caregivers, and healthcare providers. The shortcomings in user interface design, customization options, AI engagement, and recognition of real-world communication patterns underscore the urgent demand for a more holistic, adaptive, and intuitive technology—one that truly enhances seniors' quality of life and reduces caregiver burden.

### Need for an Improved Solution

There is a growing need for a comprehensive care system that addresses the full spectrum of senior support, from daily routine management to meaningful social interaction. Current solutions often overlook crucial aspects such as user interface familiarity, personalized AI engagement, and structured data collection. By offering greater versatility and user-centric design, the system can better serve not only seniors but also caregivers and other stakeholders involved in their care.

Integrates seamlessly into a senior's daily routine through a familiar interface (e.g., television or tablet). To ensure high adoption and consistent use, the system should fit effortlessly into a senior's existing habits. By relying on a familiar interface—such as a television or a tablet—seniors are not forced to learn complex new technologies. This approach reduces confusion, improves adherence, and fosters greater trust and comfort in using the platform.

Offers advanced AI capabilities for conversational reminders, health checks, and companionship. An AI-driven companion can provide more than just alerts—it can hold natural, empathetic conversations tailored to the senior's emotional and cognitive needs. With conversational reminders, the system can confirm whether a senior has truly understood and followed through on an action. Beyond reminders, AI modules can conduct well-being checks, helping detect loneliness, confusion, or health anomalies, and thereby facilitating timely interventions.

Is easily managed by caregivers via user-friendly web or mobile portals, avoiding technological hurdles for seniors. A key requirement is that caregivers—who may be family members or professional aides-can easily set up and adjust the system. Intuitive web or mobile portals let caregivers schedule tasks, monitor compliance, and review recorded interactions without needing specialized training. This user-friendly approach also ensures seniors are not burdened with configuring the system themselves, which could introduce frustration or confusion.

Increases the caregiver's efficiency, and frees up time. By automating routine interactions and basic check-ins, the system allows caregivers to focus on higher-value or more pressing tasks. Time-saving interactions—like automated medication reminders and daily mood surveys—can flag potential issues early, prompting more efficient use of caregiver resources. Ultimately, this reduces the need for frequent in-person visits for minor confirmations while still maintaining a high standard of oversight.

Collects Interaction Data for Tracking Health Indicators, Emotional States, and Task Adherence. Lastly, systematic data collection is crucial for monitoring trends over time. By capturing metrics such as medication compliance, vital sign reports, and emotional cues, caregivers and healthcare providers gain valuable insights. This information can be used to customize care plans, predict emerging issues, and enable evidence-based decision-making—resulting in more targeted, proactive care for seniors.

The present invention addresses these requirements by combining a task and reminder scheduling system with an AI-driven companion interface. Through the use of simple, easy-to-use display devices and advanced AI technology, the system substantially enhances the quality of care, ease of use, and overall user experience for older adults. Caregivers benefit from streamlined oversight and actionable data, helping them intervene when necessary and allowing seniors to maintain greater independence in their daily lives.

### SUMMARY OF THE INVENTION

The present invention provides a flexible, display-based system (e.g., television, tablet, or other suitable screens) for managing tasks, reminders, and AI-driven interactions specifically designed to improve the daily lives of seniors. By leveraging easy-to-use endpoints, advanced AI features, and a cloud-based scheduling infrastructure, this invention addresses the complex needs of older adults, caregivers, and healthcare providers.

### Task Scheduling & Execution

A cloud-based scheduling function periodically checks a central database to identify tasks due for execution (e.g., medication reminders, daily surveys). When a task is found, the system dispatches commands to one or more endpoints—such as a TV-connected device or a tablet—turning on the display if needed and presenting the task at the scheduled time. This automated process ensures that seniors receive prompts in a timely manner, reducing the risk of overlooked appointments or reminders and minimizing caregiver overhead.

### AI Companion Integration

At the core of the system is an AI companion powered by Large Language Models (LLMs). This AI component can deliver personalized interactions, ranging from medication reminders and general well-being checks to casual conversations that help reduce loneliness. In some embodiments, the AI companion also:
i. Provide medication reminders, health checks, and casual conversations. Health checks can be in the form of surveys, video recordings, or quantifiable answers (“I've had 3 glasses of water so far today”).
ii. Adapts Dialogue Dynamically: Conversations evolve based on user responses, personal preferences, and historical data. It adapts its dialogue based on collected data, and ongoing conversation context.
iii. Accepts Voice and Camera Inputs: This hands-free functionality accommodates users with mobility or dexterity challenges, enabling natural speech and possibly visual recognition for more context-aware interactions.
iv. Responds to User Commands: Seniors may ask questions, request content (e.g., music, weather updates), or seek clarifications about health.

### Caregiver Management

Caregivers or authorized users access the system through web or mobile portals, where they can create, edit, or schedule tasks and AI-based interactions. Tasks can be configured as one-time events or set to recur daily, weekly, or according to custom intervals. By centralizing these settings in a single database, caregivers can quickly review upcoming schedules, make adjustments in real time, and ensure that seniors remain engaged with minimal effort.

### Data Collection & Analysis

Every interaction—whether a conversation with the AI, a reminder confirmation, or a health survey completion—is logged and stored in the system's central database. This data can include essential health metrics, self-reported emotional states, and other indicators relevant to senior well-being. Caregivers and healthcare providers can then review aggregated reports, detect concerning trends or “red flags” (such as repeated missed medications), and modify care plans as needed. By maintaining these records over time, the system enables more personalized, data-driven support.

### Extensibility

To accommodate a variety of senior care scenarios, the invention ships with a library of pre-written tasks, including meal confirmations, daily health checks, friendly visits, and telehealth connections. Moreover, the system provides a straightforward method for adding new tasks through custom LLM prompts, ensuring it can adapt to evolving requirements. For example, a caregiver might define a specialized check-in for cognitive exercises or add new conversation topics to align with a senior's changing interests.

In one embodiment, the solution leverages a familiar television interface to deliver a non-intrusive means of communication and interaction that seniors are more likely to adopt. In other embodiments, the invention incorporates tablet-based or other portable displays for increased flexibility and mobility. Collectively, these features work in concert to improve care quality, reduce caregiver burden, and enhance seniors'daily experiences by providing comprehensive oversight and robust AI-driven assistance.

### BRIEF DESCRIPTION OF DRAWINGS

FIG. 1 is a schematic diagram illustrating the overall system architecture and data flow for providing AI-driven task management and reminders to seniors via a television-, tablet-, or other display-based interface. The labeled components include:

### CAREGIVER UI (MOBILE (104a-104c) OR WEB (103)

This represents a series of user-friendly platforms—such as mobile apps running on smartphones (104a-104c) and a web dashboard (103)—through which caregivers can create, edit, and schedule tasks or AI interactions. These interfaces communicate with the system over the internet, allowing caregivers to log in from virtually anywhere. In addition to basic scheduling functions, they may present real-time updates (e.g., confirming whether a reminder was acknowledged) and display historical data or analytics about the senior's engagement.

### TASKS & INTERACTIONS DATABASE (105)

A central repository for storing all scheduled tasks, user preferences, system configuration data, and any logs of completed interactions. Each time a caregiver creates or modifies a task, the details (e.g., timing, task type, associated AI prompt) are recorded here. By centralizing all data, the system can maintain a consistent, synchronized view of what needs to happen and when.

### CLOUD FUNCTION/SCHEDULER (106)

This scheduling component (which may be a CRON job or a dedicated serverless function) periodically queries the database (105) to identify tasks that are due for execution. When a match is found—e.g., a medication reminder scheduled for 10:00 AM—it dispatches commands to the relevant endpoint device. The scheduler thus serves as the “heartbeat” of the system, ensuring tasks occur at the proper intervals without requiring caregiver intervention.

### TV-CONNECTED DEVICE (100, 101)

A hardware interface that links directly to the senior's television, such as a set-top box, streaming stick, or dedicated controller. When the scheduler signals a task, this device powers on the TV if it is off (100 shows the camera/microphone, while 101 illustrates a separate hardware module including cpu, memory, hdmi connectors, etc. These may be in the same physical package). It then displays or executes the scheduled task (e.g., a health check or video call). Once finished, the TV is returned to its normal programming or powered off if desired.

### TABLET DEVICE (102)

An alternative (or additional) endpoint for delivering tasks and interactions, especially useful for seniors who may not be in front of a TV and need a portable device. Like the TV-connected device, the tablet “wakes up” when a scheduled task arrives, presents the reminder or AI-driven conversation, and then reverts to its default state when completed. This flexibility allows seniors to receive prompts whether they are in front of their television or bed-ridden. The tablet wakes and displays or executes tasks, reverts to normal operation afterwards.

### AI COMPANION MODULE (107)

A software module powered by Large Language Models (LLMs). This module processes both incoming data (such as user speech or prior interaction logs) and outgoing content (such as dynamic prompts, follow-up questions). By using the LLM's natural language capabilities, the AI companion can engage in voice or text-based conversations, adapt its responses to each senior's preferences, and provide a more empathetic, human-like experience.

### TEXT TO SPEECH/SPEECH TO TEXT (108)

This subsystem facilitates two-way voice interaction by converting the AI companion's textual responses into audible speech (Text-to-Speech) and interpreting seniors'spoken words as text (Speech-to-Text). This functionality is critical for those with limited dexterity or vision, enabling them to respond verbally rather than navigating on-screen menus. In some embodiments, the engine may also handle different languages, dialects, or specialized vocabularies for seniors with unique communication needs.

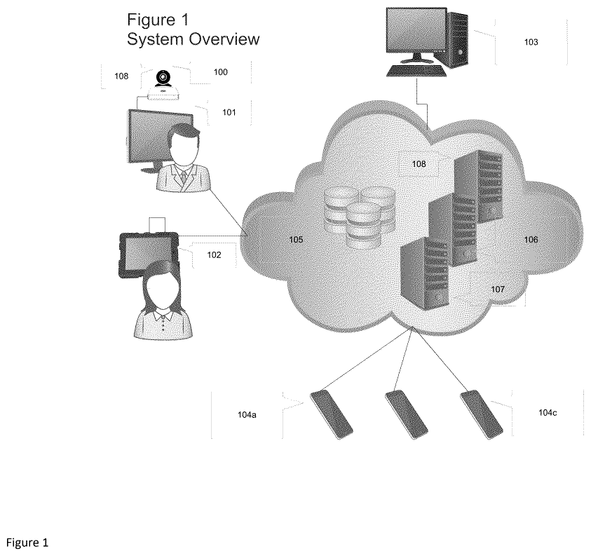

Each labeled component in FIG. 1 works in tandem to provide a seamless experience for seniors and caregivers. The diagram illustrates how a caregiver's commands travel from the UIs (103, 104a-104c) through the cloud-based scheduling logic (106), triggering tasks on the chosen endpoint device (100, 101, 102), which then coordinates with the AI Companion (107) and voice processing engines (108) to deliver context-aware and personalized support. By centralizing scheduling, data storage, and AI processing, the system ensures consistent, real-time management of tasks, reminders, and interactions.

### DETAILED DESCRIPTION OF THE INVENTION

### System Architecture

The system comprises various components working in concert to deliver scheduled tasks, reminders, and AI-driven interactions. Each component is outlined below:

Caregiver/Administrator User Interfaces: (103, 104a-104c)

This includes both mobile apps and web-based portals, through which caregivers can create, schedule, and manage tasks, as well as configure AI interaction preferences. By logging in through a smartphone (e.g., 104a-104c) or a browser-based console (103), caregivers can remotely add new reminders, monitor completed tasks, and adjust system settings in real time. Each UI communicates with the backend services over the internet, ensuring seamless updates and consistent data across all devices.

### Central Database: (105)

The database stores task schedules, user profiles, senior preferences, and conversation logs, acting as the single source of truth for all system interactions. For each scheduled task, the database captures metadata such as timing, frequency, and any specialized AI prompts to be used. Additionally, it maintains individualized records—like a senior's previous task outcomes, conversation transcripts, and any caregiver-generated notes—supporting personalized and context-aware AI responses.

### Scheduled Task Runner (Cloud Function) (106)

A serverless function or dedicated scheduler (e.g., running as a CRON job) periodically checks the database (105) for tasks that are due. Operating at a set interval, such as once per minute, the task runner identifies which seniors have tasks that need executing at that exact time. Upon finding relevant tasks, it dispatches commands to the appropriate endpoint devices, thus automating the entire delivery and ensuring that caregivers do not have to manually trigger reminders.

### TV-Connected Endpoint Devices (100,101)

These physical units can be specialized hardware boxes, streaming sticks, or set-top devices designed to interface directly with a television. They include or have access to microphone and camera inputs (100) so seniors can interact with the system via voice and (optionally) video. When a scheduled task arrives, the device (101) turns the TV on (if off), displays the scheduled prompt or AI-driven conversation, and reverts to regular programming once the interaction is complete.

### Other Display Endpoint Devices (102)

In addition to the TV, the system supports tablets, mobile devices, or other portable displays connected to the internet. For seniors who do not have a television, do not want to use their television or need the display in a different room than the television, these endpoints offer the same set of AI capabilities—microphone/camera interaction, visual prompts, and scheduled reminders—as the TV-connected hardware. They “wake” when a task is due and revert to normal operation afterward.

### AI Companion (107)

A software module powered by Large Language Models (LLMs) that drives the system's conversational capabilities. This AI companion can parse both text-and voice-based inputs, adapting its responses based on stored data (e.g., user preferences, past interactions). By leveraging advanced natural language understanding, it can maintain context, remember previous conversations, and address tangential queries while remaining focused on its primary task.

### TTS/STT (108)

Text-to-Speech (TTS) and Speech-to-Text (STT) components are crucial for natural voice interaction. They may be located within the endpoint device, on a tablet, or in the cloud. TTS converts the AI companion's text responses into audible speech, while STT interprets the senior's spoken words, enabling the AI companion to respond to user commands and questions fluidly without requiring any manual text input.

### Task Scheduling and Execution Flow

### Task Creation

Caregivers log into the UI (mobile or web) and use a “Create Routine” feature to define the time, recurrence, and task type (e.g., a medication reminder, AI conversation, telehealth appointment, etc.). They can also attach specific AI prompts—such as a script for a survey—to guide the AI companion in how it should interact when that task is triggered.

### Database Storage

All newly created tasks are saved to the central database (105). If the task involves an AI conversation, the relevant LLM prompts or scripts are stored as well. By centralizing this information, the system can keep track of which tasks are associated with each senior, ensuring that subsequent steps reference the correct data.

### Scheduler Detection

A cloud function (106) runs continuously at fixed intervals—commonly once a minute—to see whether any tasks are due at the current time. If it finds tasks that are ready, the scheduler compiles a list of the target seniors, the endpoint devices they use, and any relevant parameters (e.g., content to display, voice prompts, or conversation scripts).

### Device Instruction

For each relevant task, the system sends a real-time command to the corresponding device—whether it's the TV-connected endpoint (101) or a tablet (102). These commands may include instructions to power on the display, begin streaming video or audio, or initiate a conversation through the AI companion.

TV Activation & Task Execution—(Optional for TV displays)

When tasks are directed to the TV-connected endpoint, the device automatically powers on the TV (if it is currently off), then displays the reminder or conversation. For example, a medication prompt might appear on screen accompanied by a voice message: “Hello, it's 8 AM; please remember to take your morning pills.” After user interaction, the device returns the TV to its normal state, such as live TV or powered off.

### Tablet Task Execution—(Optional for Other Displays)

Alternatively, tasks may be pushed to a tablet endpoint (102). Here, the device might wake from sleep mode, show a pop-up notification, and initiate an AI-driven conversation—for example: “Good afternoon, have you had lunch yet?” This approach is especially convenient when seniors prefer a separate screen rather than their television.

### User Interaction

The senior typically responds via voice (and optionally camera), allowing the AI companion (107) to record data or adapt its conversational flow. If the task is a well-being check, the user might speak answers like “Yes, I'm feeling fine,” which the AI companion logs in the database. Complex prompts (e.g., “On a scale from 1 to 10, how would you rate your pain?”) can also be handled seamlessly by capturing numeric or free-form replies.

### Completion & Reversion

Once the task concludes, the device transitions back to its normal functionality. A television endpoint might resume the previously viewed channel, while a tablet may return to its home screen or lock screen or a convenience function like a photo frame. This design ensures minimal disruption for seniors, reducing frustration and making it easier for them to continue their normal routine.

### AI Companion Features

### LLM-Based Dialogues

Tasks that involve conversational interaction rely on advanced LLM prompts to shape the AI companion's demeanor, questions, and responses. The LLM can interpret free-form answers, ask clarifying questions, and maintain coherence throughout longer discussions. This is crucial when seniors respond in non-standard ways or offer more context than a simple “yes” or “no.” The LLM can respond intelligently to follow-up questions, tangential discussions, or clarifications while keeping focus on the main task.

### Data Logging

Every user utterance, AI response, or system prompt can be captured in conversation transcripts. These logs are added to the central database and may include timestamps, recognized keywords, or sentiment analysis flags (e.g., “User sounded distressed”). Caregivers can later review these logs to identify patterns or red flags, like repeated complaints of pain, missed medication times, or changes in mood. Every event is also considered data and the time and type of event is logged to also keep a record of the seniors level of engagement.

### Voice & Video Integration

The system leverages microphone input to facilitate natural voice interactions, enabling seniors to talk to the AI companion as though they were speaking to a person. Meanwhile, a camera may be employed for telehealth tasks—such as capturing images of medication labels, checking physical well-being, or enabling a face-to-face feeling of connection. All video usage is subject to privacy regulations and user consent, ensuring compliance with healthcare standards.

### Adaptive Prompts

Using previously gathered data and context, the AI companion can modify its prompts in real time. For instance, if the senior regularly reports difficulty remembering dinner routines, the system might schedule more frequent evening check-ins or offer structured reminders. This adaptive approach fosters higher engagement and better addresses each senior's evolving care needs.

### Data Collection, Analysis, and Alerts

### Structured Surveys

The invention supports scripted surveys or assessments aimed at collecting daily or periodic user data—ranging from basic mood checks to more detailed health questions (e.g., “Did you measure your blood pressure today?”). Responses are stored for further analysis and may prompt immediate follow-ups if necessary.

### Analysis & Alerts

If the system detects adverse trends—for example, multiple missed medications or repeated self-reports of pain—it can automatically notify caregivers or initiate a telehealth session. This helps caregivers intervene proactively, potentially averting more serious health incidents.

### Caregiver Dashboard

A dedicated dashboard consolidates daily summaries of the senior's compliance, well-being indicators, and key highlights from AI interactions. Caregivers gain a real-time overview of potential issues, making it easier to coordinate care and schedule in-person visits only when truly needed.

### Extensibility

### Pre-Defined Tasks

Common senior care tasks—like meal reminders, daily check-ins, medication tracking—come built-in as templates. These ready-made tasks can be activated with minimal setup, ensuring caregivers or administrators can start using the system immediately without scripting every new interaction from scratch.

### Custom LLM Prompts

Should a caregiver need more specialized tasks—for example, checking a senior's reaction to a newly prescribed medication—they can create custom LLM prompts. This allows the AI companion to ask specific questions, track unique data points, or adapt to specialized care routines, all without extensive coding or system overhauls. Over time, these custom prompts can evolve into new templates or best practices, further enriching the platform's functionality.

1-6. (canceled)

7. A system for integrating health monitoring and AI-driven companionship for a patient via multiple device form factors, comprising:
a. means for capturing daily health indicators through voice or camera-based prompts, storing said indicators in the database, and associating them with the patient's profile for longitudinal analysis;
b. means for generating alerts or notifications to one or more caregivers if certain thresholds are exceeded, multiple tasks are missed, or a negative trend is detected, allowing proactive intervention;
c. a caregiver dashboard providing aggregated insights into daily routines, compliance rates, emotional well-being indicators, and conversation highlights, facilitating data-driven care decisions; and
d. an endpoint device wherein patient may choose a television interface or a tablet-based interface for receiving reminders, completing tasks, and engaging with an AI companion, wherein the AI companion calls the patient to collect health information, or provide verbal companionship.

8. The system of claim 7, wherein the means for capturing daily health indicators comprises a scripted survey presented by the AI companion that prompts the patient to report at least one of: medication intake, pain level, mood, hydration level, or completion of a meal.

9. The system of claim 7, wherein the means for capturing daily health indicators further comprises structured surveys configured for periodic administration, the surveys including questions such as whether the patient has measured blood pressure that day and a request for a self-reported numeric value of the blood pressure.

10. The system of claim 7, wherein the means for capturing daily health indicators is further configured to log free-form verbal responses from the patient and extract structured data therefrom using the AI companion module.

11. The system of claim 7, wherein the means for capturing daily health indicators includes a conversational AI interface.

12. The system of claim 7, wherein the means for capturing daily health indicators further comprises a camera configured to capture still images or video clips during telehealth tasks to permit visual assessment of the patient's well-being.

13. The system of claim 7, wherein the means for capturing daily health indicators associates each captured indicator with a timestamp and a task identifier in the database, thereby enabling longitudinal trend analysis.

14. The system of claim 7, wherein the means for generating alerts or notifications to caregivers is configured to automatically issue an alert when multiple scheduled medication-related tasks are missed within a predefined time window.

15. The system of claim 7, wherein the means for generating alerts or notifications to caregivers further comprises logic configured to detect repeated self-reports of pain above a threshold level and, in response, notify a caregiver or initiate a telehealth session.

16. The system of claim 7, wherein the means for generating alerts or notifications to caregivers is configured to monitor sentiment indicators derived from conversation transcripts and to generate an alert when sustained negative sentiment is detected over multiple interactions.

17. The system of claim 7, wherein the caregiver dashboard is configured to display aggregated compliance metrics including at least one of: percentage of completed medication reminders, number of missed tasks per day, and frequency of caregiver-initiated interventions.

18. The system of claim 7, wherein the caregiver dashboard further presents visual summaries of emotional well-being indicators, including trend lines of self-reported mood or sentiment scores derived from conversation logs.

19. The system of claim 7, wherein the caregiver dashboard is configured to surface conversation highlights that match predefined red-flag criteria, including user utterances indicating confusion, distress, loneliness, or requests for medical assistance.

20. The system of claim 7, wherein the endpoint device flexibility comprises a television-connected endpoint configured to automatically power on a television at a scheduled task time, present an AI-driven interaction, and return the television to a prior state upon completion of the interaction.

21. The system of claim 7, wherein the endpoint device flexibility further comprises a tablet endpoint configured to wake from a sleep state upon receipt of a scheduled task, present a notification and conversational prompt, and return to a default home or lock screen upon completion.

22. The system of claim 7, wherein the endpoint device flexibility allows a single patient profile to be associated concurrently with both a television endpoint, a tablet endpoint, or telephone endpoint, and wherein tasks can be selectively routed either endpoints, or all simultaneously based on configuration data in the database.

23. The system of claim 7, wherein the AI companion is configured to adapt the content and frequency of health-indicator prompts based on historical compliance data stored for the senior.

24. The system of claim 7, wherein the AI companion is configured to modify its conversational tone or follow-up questions when prior interaction logs indicate that the senior frequently provides non-standard or tangential responses.

25. The system of claim 7, wherein the means for capturing daily health indicators and the caregiver dashboard are jointly configured to support caregiver-defined custom LLM prompts such that newly defined health checks or surveys are automatically incorporated into subsequent trend and compliance views.

26. The system of claim 7, wherein the means for generating alerts or notifications to caregivers includes a rules engine stored in association with the database, the rules engine being configurable via the caregiver dashboard to specify alert thresholds for different health indicators and compliance metrics.
